(19) United States
(12) Patent Application Publication
(10) Pub. No.: US 2024/0156817 A1
(43) Pub. Date: May 16, 2024

(54) TREATMENTS FOR PAIN

(71) Applicant: Sevenless Therapeutics Limited, Canterbury Kent (GB)

(72) Inventors: Neil BENSON, Canterbury Kent (GB); Alasdair NAYLOR, Canterbury Kent (GB)

(21) Appl. No.: 18/477,958

(22) Filed: Sep. 29, 2023

Related U.S. Application Data
(63) Continuation of application No. PCT/EP2022/058337, filed on Mar. 29, 2022.

(30) Foreign Application Priority Data
Mar. 31, 2021 (GB) 2104609.9
Sep. 21, 2021 (GB) 2113435.8

(51) Int. Cl. A61K 31/517; A61K 31/4155; A61K 31/4178; A61K 31/4545; A61K 31/495; A61K 31/496; A61K 31/502; A61K 31/519; A61K 31/53; A61K 31/5377; A61K 39/395; A61P 29/02

(52) U.S. Cl. CPC A61K 31/517; A61K 31/4155; A61K 31/4178; A61K 31/4545; A61K 31/495; A61K 31/496; A61K 31/502; A61K 31/519; A61K 31/53; A61K 31/5377; A61K 39/3955; A61P 29/02; A61K 2039/505

(57) ABSTRACT

The Application describes a new target for the treatment of pain and identifies suitable compounds for use in the invention.

The present invention describes the use of compounds that bind to the Son of Sevenless homolog 1 receptor (SOS1) protein and rat sarcoma (RAS) thereby inhibiting a cascade pathway, leading to a reduction in pain.

There remains a need for novel treatments for the treatment of pain. Many of the most efficacious and frequently prescribed pain treatments are opioids. These drugs have a high potential for abuse and addiction. However, to date there are no treatments of comparable efficacy to replace them as front-line treatments. To provide a paradigm shift in the treatment of pain requires the identification of new targets and pathways within the body.

This application describes the identification and exploitation of SOS-Ras in a suitable pathway for the treatment of Pain.

SOS1 inhibitors have recently been identified capable of mediating several conditions:

WO2019/122129 describes benzylamino substituted pyridopyrimidines as SOS1 inhibitors useful in the treatment of cancerous growth in oncology.

WO2018/115380 describes benzylamino substituted quinazolines as SOS1 inhibitors, similarly useful in the treatment of cancerous growth in oncology.

WO2018/172250 describes a genus of 2 methyl quinazolines for use in treating hyper-proliferative diseases.

WO2019/201848 describes a further genus of 2 methyl quinazolines for use in treating hyper-proliferative diseases.

WO2020/173935 teaches new isoindolinone substituted indoles as RAS inhibitors.

Further SOS1 inhibitors are taught in Proceedings of the National Academy of Sciences of the United States of America (2019), 116(7), 2551-2560.

Various RAS inhibitors including a subset of Ras inhibitors known as KRAS have also recently been identified. WO2018/068017, WO2018/140513, WO2018/140514 & WO2020/173938 all teach new compounds with activity as RAS inhibitors to treat cancer. A summary of new Ras inhibitors and their clinical status may be found at RSC Med. Chem., 2020, 11, 760.

Surprisingly it has now been found that the SOS1/Ras pathway can be exploited for the treatment of pain.

Accordingly, the present invention provides SOS1 inhibitors for use in the treatment of Pain.

The present invention also provides RAS inhibitors for use in the treatment of Pain.

### FIGURES

FIG. 1. NGF signal transduction pathway leading to pain and the clinical drugs that validate the pathway. NGF binds to TrkA and subsequent signal transduction culminates in the nuclear accumulation of diphopshorylated Extracellular signal-regulated kinase (dppERKnuc) in neurons, upregulating pain genes.

FIG. 2. Clinical genetic validation of the target. In NF1, patients have a mutation in the neuronal gap protein (NF1). The mutation causes a loss of function, preventing the normal turnover of GTP on RAS_{GTP} to GDP in turn increasing the concentration of RAS_{GTP} and leading to excess signalling, tumours and pain.

FIG. 3 Graph of results from Inhibition of Nerve Growth Factor (NGF) stimulated phospho-Extracellular Regulated Kinase 1 and 2 (pERK1/2) activation in the PC-12 cell line by BI3406.

FIG. 4 Graph showing the effects of BI3406 at 25 mg/kg (10 mL/kg, p.o., am and pm dosing, 2 days), in combination with a single dose of Tanezumab at 0.1, 0.3 and 1 mg/kg (10 mL/kg, i.p., T=0) and Tanezumab alone at 3 mg/kg (10 mL/kg, i.p., T=0) using weight-bearing (WB) assessments at 1 h post-dosing for BI3406 and +1 h, +9 h, +25 h and +33 h post Tanezumab administration.

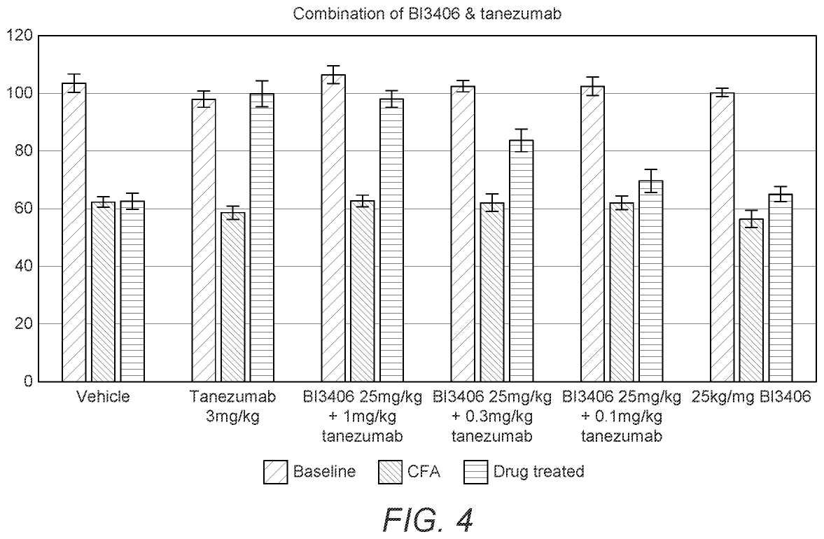

### SUMMARY OF INVENTION

This application describes the identification and exploitation of the SOS-Ras target as appropriate pathways for the treatment of Pain.

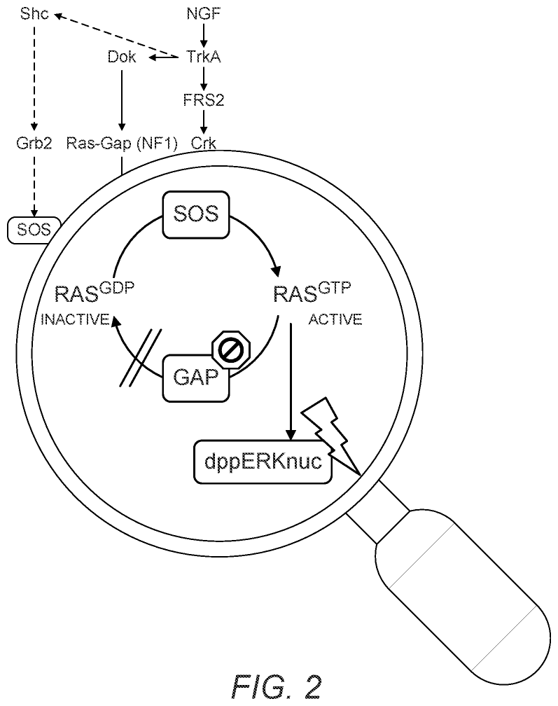

Ras proteins are known to be a key element in the maintenance of tumours and so the target has long been considered attractive in oncology. However, until recently SOS1-Ras was seen as an undruggable target. The canonical property of Ras is that of a small GTPase which normally cycles between a GTP-bound active state and a GDP-bound inactive state, facilitated in part by GTPase activating protein (GAP) stimulation of GTP hydrolysis (FIG. 2). However, when Ras proteins are mutationally activated, impaired GAP stimulation favours the formation of persistently GTP-bound Ras. This critical biochemical defect prompted the earliest efforts to target mutant Ras. By analogy to the ATP-competitive inhibitors that are effective antagonists of protein kinases, identification of GTP-competitive inhibitors of Ras has been attempted. However, whereas ATP binds protein kinases with low micromolar affinity, GTP binds Ras proteins with picomolar affinity, preventing discovery of effective inhibitors.

With the discovery of suitable SOS1 inhibitors and Ras inhibitors it has been possible to investigate the pathway for additional indications, other than cancer. Surprisingly, detailed investigation has now revealed its possible to have a positive effect on pain by using SOS1 inhibitors and Ras inhibitors.

The present invention provides SOS1 inhibitors for use in the treatment of Pain.

The activity of a SOS1 inhibitor may be measured in the HTRF binding assay described in Hillig et al, PNAS | Feb. 12, 2019 | vol. 116 | no. 7 | 2551-2560.

Other SOS1 assays are well known to the skilled person and include assays such as FRET/SPR binding.

Suitable SOS1 inhibitors for use in the present invention have an IC₅₀'s in the HTRF binding assay of less than or equal to 5 micromolar.

Particularly suitable SOS1 inhibitors have an IC₅₀ of less than 100 nanomolar in the HTRF binding assay.

In a particularly preferred embodiment, the SOS1 inhibitors have an IC₅₀ of 1 nanomolar or less in the HTRF binding assay.

The SOS1 inhibitors of the present invention also show selectivity for SOS1 over additional targets. Suitably, the SOS1 inhibitors of the present invention show selectivity of greater than or equal to 100 fold over one or more of the following targets: MEK 1, MEK 2, TrkA kinase, TrkB kinase, TrkC kinase, C-Raf, B-Raf, PI3 kinase, AKT and ERK.

When determining whether a compound of the present invention has a selectivity of more than a 100 fold for SOS1 over another target the following assays and methods may be used:

MEK 1 and 2 can be assayed using MEK assay kit, product code CS0490, Sigma, St Louis, USA.

Trk receptor kinase activity can be assayed as described in Wang et al, Curr Chem Genomics. 2008; 1: 27-33.

B-Raf can be assayed using the B-Raf Kinase Assay Kit, product code 17-359, Sigma, St Louis, USA.

C-Raf can be assayed using the BPS bioscience assay kit catalogue number 79570, San Diego, CA 92121. United States.

PI3 kinase can be assayed via the method described by Fry, Methods Mol Biol, 2009;462:345-62.

AKT can be assayed using the abcam kit Akt Kinase Activity Assay Kit (ab139436), abcam plc, Cambridge, USA.

ERK can be assayed using the Promega ERK2 kinase kit, catalogue number V1961, Promega corporation, Madison, USA.

Suitable SOS1 inhibitors include those disclosed in WO2019/122129, WO2018/115380 and WO2018/172250, WO2019/201848 and WO2020/173935

Further SOS1 inhibitors are taught in Proceedings of the National Academy of Sciences of the United States of America (2019), 116(7), 2551-2560.

Indoles as described in; ACS Med Chem Lett 9 (9), 941-946 (2018).

Indoles as described in; J Med Chem 61 (19), 8875-8894 (2018).

Benzimidazoles as described in; J Med Chem 61 (19), 8875-8894 (2018)

Cancer Discovery, 2020 Aug 19;CD-20-0142. doi: 10.1158/2159-8290.CD-20-0142.

Lu et al, Chem MedChem 2016, 11, 814 — 821.

Particularly suitable compounds are:

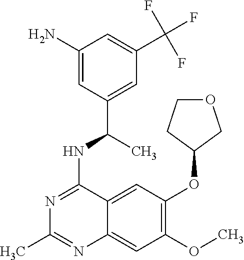

BI 3406 (N-[(1R)-1-[3-amino-5-(trifluorornethyl)phenyl]ethyl]-7-methoxy-2-methyl-6-[(3S)-oxolan-3-yl]oxyquinazolin-4-amine)

Synthesis of this molecule is published in WO2018115380

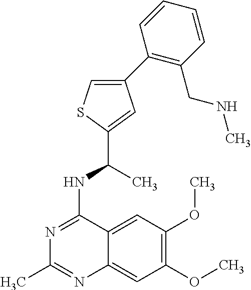

Bay 293 (6,7-dimethoxy-2-methyl-N-[(1R)-1-[4-[2-(methylaminomethyl)phenyl] thiophen-2-yl]ethyl]quinazolin-4-amine). Synthesis published in PNAS 2019 116 (7) 2551-2560

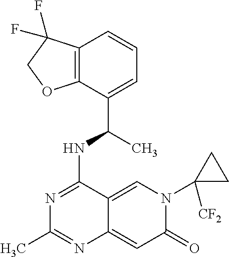

4[[(1R)-1-(3,3-difluoro-2H-1-benzofuran-7-yl)ethyl]amino]-6-[1-(difluoromethyl)cyclopropyl]-2-methylpyrido[4,3-d]pyrimidin-7-one. Synthesis published in WO2019122129.

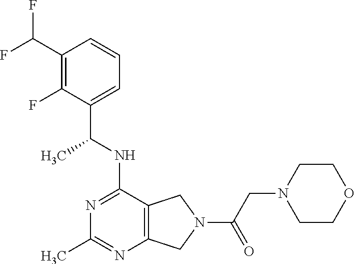 [SMILES: Cc1nc2c(c(N[C@H](C)c3cccc(C(F)F)c3F)n1)CN(C(=O)CN1CCOCC1)C2]

Synthesis published in WO2020180770

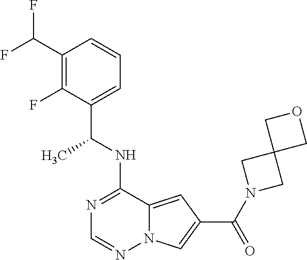 [SMILES: C[C@@H](Nc1ncnn2cc(C(=O)N3CC4(COC4)C3)cc12)c1cccc(C(F)F)c1F]

Synthesis published in WO2020180768;

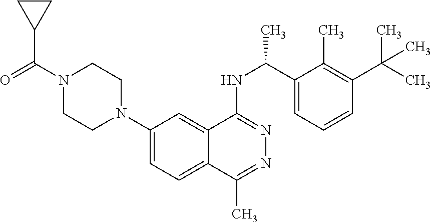 [SMILES: Cc1c([C@@H](C)Nc2nnc(C)c3ccc(N4CCN(C(=O)C5CC5)CC4)cc23)cccc1C(C)(C)C]

(R)-cyclopropyl(4-(1-methyl-4-((1-(2-methyl-3-(frifluromethyl)phenyl)ethyl)amino)phthalazine-6-yl)piperazin-1-yl)methanone

Synthesis published in WO2021127429

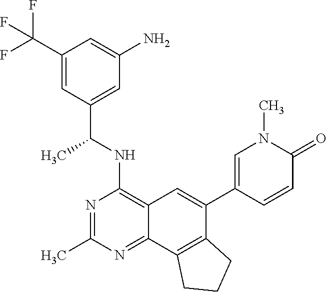 [SMILES: Cc1nc(N[C@H](C)c2cc(N)cc(C(F)(F)F)c2)c2cc(-c3ccc(=O)n(C)c3)c3c(c2n1)CCC3]

(R)-5-(4-((1-(3-amino-5-(trifluoromethyl)phenypethypamino)-2-methyl-8,9-dihydro-7H-cyclopenta[h]quinazolin-6-yl)-1methylpyridin-2(1H)-one

Synthesis published in WP2021105960

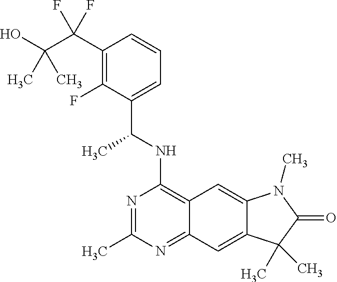 [SMILES: Cc1nc(N[C@H](C)c2cccc(C(F)(F)C(C)(C)O)c2F)c2cc3c(cc2n1)C(C)(C)C(=O)N3C]

(R)-4-((1-(3-(1,1-difluoro-2-hydroxy-2-methylpropyl)-2-fluorophenypethypamino)-2,6,8,8-tetramethyl-6,8-dihyro-7H-pyrrolo[2,3-g]quinazolin-7-one

Synthesis published in WO2021130731; and

BI-170963 disclosed WO2019122129.

Particularly suitable compounds are

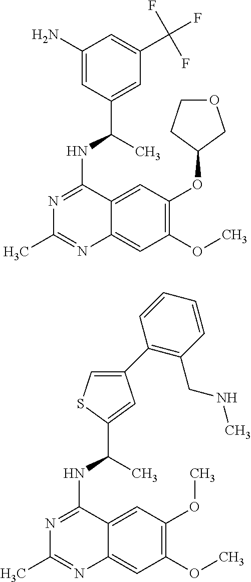

The SOS1 inhibitors were tested in an in vitro model of pain, on the NGF stimulated PC12 assay (Sasagawa et al, NATURE CELL BIOLOGY VOLUME 7, NUMBER 4, APRIL 2005, 365-373). The tested compounds showed great efficacy in the model. In particular BI3406 showed efficacy comparable with the pain drug candidate Tanezumab.

The SOS1 inhibitors have numerous advantages as a pain treatment; they don't have the addiction potential of opiates and they show great efficacy. They also don't appear to have the side effects that make tanezumab and other anti-NGFs almost impossible to use at therapeutically effective doses.

The present invention provides Ras inhibitors for use in the treatment of Pain.

The activity of a Ras inhibitor may be measured in the binding assay described by Kessler et al, Proc Natl Acad Sci USA. 2019 Aug 6; 116(32): 15823-15829.

Suitable Ras inhibitors for use in the present invention have an IC₅₀ in the binding assay of less than or equal to 5000 nM.

Particularly suitable Ras inhibitors have an IC₅₀ of less than 100 nanomolar.

In a particularly preferred embodiment, the Ras inhibitors have an IC₅₀ of 1 nanomolar or less.

The Ras inhibitors of the present invention also show selectivity for Ras over additional targets. Suitably, the Ras inhibitors of the present invention show selectivity of greater than or equal to 100 fold over one or more of the following targets: MEK 1, MEK 2, TrkA kinase, TrkB kinase, TrkC kinase, C-Raf, B-Raf, PI3 kinase, AKT and ERK

There are a number of Ras inhibitors currently in development; Araxes compounds ARS-3248, ARS-1620 & ARS-853, Amgen's AMG-510, Mirati's MRTX-849 and BridgeBio's BBP-454 have all been investigated as cancer drugs. Such Ras inhibitors would be suitable for use in the present invention.

### Other Ras Inhibitors Include:

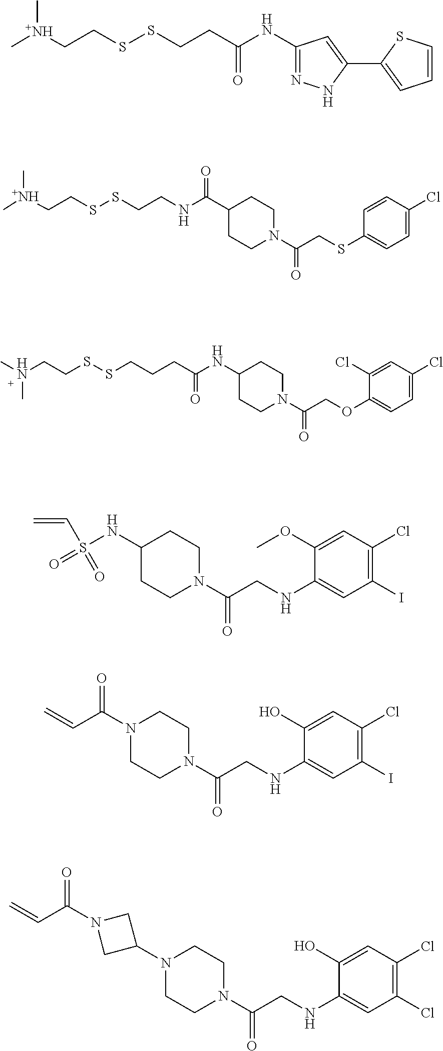

Their synthesis maybe found at J. M. Ostrem, U. Peters, M. L. Sos, J. A. Wells and K. M. Shokat, Nature, 2013, 503, 548-551; and at

M. P. Patricelli, M. R. Janes, L. S. Li, R. Hansen, U. Peters, L. V. Kessler, Y. Chen, J. M. Kucharski, J. Feng, T. Ely, J. H. Chen, S. J. Firdaus, A. Babbar, P. Ren and Y. Liu, Cancer Discovery, 2016, 6, 316-329.

### Particularly Suitable Ras Inhibitors Include:

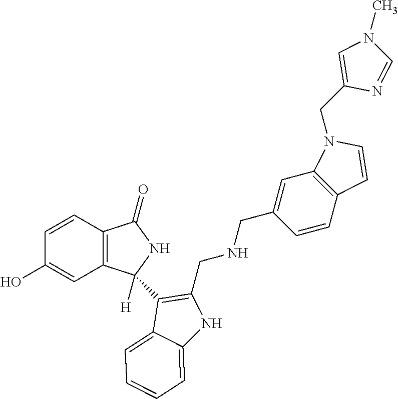 [SMILES: Cn1cnc(Cn2ccc3ccc(CNCc4[nH]c5ccccc5c4[C@H]4NC(=O)c5ccc(O)cc54)cc32)c1]

### BI-2852

(3S)-5-hydroxy-3-[2-[[[1-[(1-methylimidazol-4-yl)methyl]indol-6-yl]methylamino]methyl]-1H-indol-3-yl]-2,3-dihydroisoindol-1-one

ARS-3248/JNJ74699157

ARS-1620

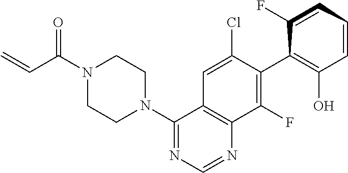

M. R. Janes, J. Zhang, L. S. Li, R. Hansen, U. Peters, X. Guo, Y. Chen, A. Babbar, S. J. Firdaus, L. Darjania, J. Feng, J. H. Chen, S. Li, S. Li, Y. O. Long, C. Thach, Y. Liu, A. Zarieh, T. Ely, J. M. Kucharski, L. V. Kessler, T. Wu, K. Yu, Y. Wang, Y. Yao, X. Deng, P. P. Zarrinkar, D. Brehmer, D. Dhanak, M. V. Lorenzi, D. Hu-Lowe, M. P. Patricelli, P. Ren and Y. Liu, Cell, 2018, 172, 578-589.e517.

ARS-853

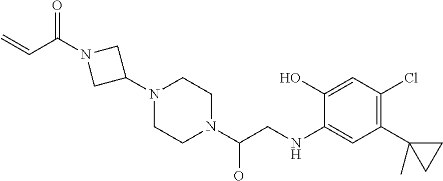 [SMILES: C=CC(=O)N1CC(N2CCN(C(O)CNc3cc(C4(C)CC4)c(Cl)cc3O)CC2)C1]

M. P. Patricelli, M. R. Janes, L. S. Li, R. Hansen, U. Peters, L. V. Kessler, Y. Chen, J. M. Kucharski, J. Feng, T. Ely, J. H. Chen, S. J. Firdaus, A. Babbar, P. Ren and Y. Liu, Cancer Discovery, 2016, 6, 316-329.

AMG-510

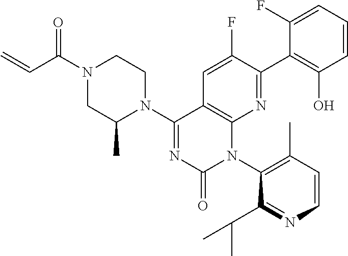

J. Canon, K. Rex, A. Y. Saiki, C. Mohr, K. Cooke, D. Bagal, K. Gaida, T. Holt, C. G. Knutson, N. Koppada, B. A. Lanman, J. Werner, A. S. Rapaport, T. San Miguel, R. Ortiz, T. Osgood, J. R. Sun, X. Zhu, J. D. McCarter, L. P. Volak, B. E. Houk, M. G. Fakih, B. H. O'Neil, T. J. Price, G. S. Falchook, J. Desai, J. Kuo, R. Govindan, D. S. Hong, W. Ouyang, H. Henary, T. Arvedson, V. J. Cee and J. R. Lipford, Nature, 2019, 575, 217-223.

MRTX-849

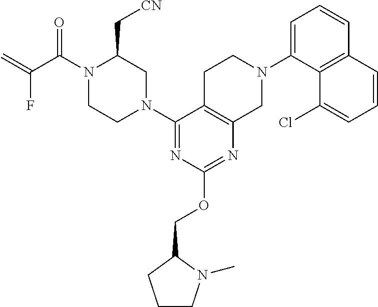

J. Hallin, L. D. Engstrom, L. Hargis, A. Calinisan, R. Aranda, D. M. Briere, N. Sudhakar, V. Bowcut, B. R. Baer, J. A. Ballard, M. R. Burkard, J. B. Fell, J. P. Fischer, G. P. Vigers, Y. Xue, S. Gatto, J. Fernandez-Banet, A. Pavlicek, K. Velastagui, R. C. Chao, J. Barton, M. Pierobon, E. Baldelli, E. F. Patricoin 3rd, D. P. Cassidy, M. A. Marx, I. I. Rybkin, M. L. Johnson, S. I. Ou, P. Lito, K. P. Papadopoulos, P. A. Janne, P. Olson and J. G. Christensen, Cancer Discovery, 2020, 10, 54-71.

J. B. Fell, J. P. Fischer, B. R. Baer, J. F. Blake, K. Bouhana, D. M. Briere, K. D. Brown, L. E. Burgess, A. C. Burns, M. R. Burkard, H. Chiang, M. J. Chicarelli, A. W. Cook, J. J. Gaudino, J. Hallin, L. Hanson, D. P. Hartley, E. J. Hicken, G. P. Hingorani, R. J. Hinklin, M. J. Mejia, P. Olson, J. N. Otten, S. P. Rhodes, M. E. Rodriguez, P. Savechenkov, D. J. Smith, N. Sudhakar, F. X. Sullivan, T. P. Tang, G. P. Vigers, L. Wollenberg, J. G. Christensen and M. A. Marx, J. Med. Chem.,

2020, DOI: 10.1021/acs.jmedchem.9b02052.

And BBP-454

Additional Ras inhibitors are described in RSC Med. Chem., 2020, 11, 760

In a further embodiment of the present invention, SOS1 inhibitors have been found to be particularly suitable for use in the treatment of pain when administered in combination with an anti NGF antibody.

In a further embodiment of the present invention, Ras inhibitors have been found to be particularly suitable for use in the treatment of pain when administered in combination with an anti NGF antibody.

The present invention provides a method of treating pain by administering a therapeutically effective amount of a SOS1 inhibitor in combination with an anti-NGF antibody.

Tanezumab is an example of an anti-NGF antibody. Its a promising and highly efficacious pain therapy, but patients frequently suffer unpleasant side effects at dosage levels sufficient to provide pain relief.

The combination provides a cooperative level of efficacy, with the advantage that the anti-NGF antibody can be administered at a dosage levels sufficient to provide pain relief without reaching a level where an adverse event may be seen. In cooperative systems, two independent agents are able to show a level of activity equivalent to one of the agents at a much higher dose. Its surprising to find two agents combining to have such an effect.

The scientific literature teaches that Tanezumab shows efficacy in rats at 10 mg/kg. (Miyagi et al Efficacy of nerve growth factor antibody in a knee osteoarthritis pain model in mice https://www.ncbi.nlm.nih.gov/pmc/articles/PMC5670727/ Max efficacious dose of anti NGF is 10 mg/kg mouse Ghilardi et al Neuroplasticity of Sensory and Sympathetic Nerve Fibers in the Painful Arthritic Joint https://www.ncbi.nlm.nih.gov/pmc/articles/PMC3386465/ Max efficacious dose of anti NGF is 10 mg/kg mouse Shelton et al Nerve growth factor mediates hyperalgesia and cachexia in auto-immune arthritis https://pubmed.ncbi.nlm.nih.gov/15927377/ Max efficacious dose of anti NGF is 10 mg/kg mouse)

BI3406 has been shown to have efficacy in a pain model in mice at a dose of 10 mg/kg.

Dosing the SOS1 inhibitor BI3406 at 25 mg/kg has been shown to demonstrate little to no efficacy in a pain model.

Surprisingly, low doses of a SOS1(25 mg/kg) 813406 in combination with 0.3 mg/kg Tanezumab has a significant analgesic effect. Low doses of a SOS1(25 mg/kg) BI3406 in combination with 1.0 mg Tanezumab has a significant analgesic effect The increase in analgesia with fixed dose SOSi is related to the dose of Tanazumab (ie efficacy is dose related)

These data indicate that it should be possible to lower the dose of Tanezumab in humans and as a result reduce the propensity for side effects that limit use if of this class of drug. The combination of SOS1 inhibition with NGF blocking via monoclonal antibodies such as Tanezumab will deliver increased pain efficacy with reduced side effects when compared to the use of higher doses of Tanezumab alone.

Combinations of SOS inhibitors with NGF monoclonal antibodies, or other blockers/modulators of the NGF pathway, have the potential to deliver greater pain efficacy with reduced side effects leading to improved and enhanced treatment of pain in conditions such as osteoarthritis.

Accordingly, the present invention provides for the use of a SOS1 inhibitor in combination with an anti NGF, wherein one or both components is administered at a sub-therapeutic dose for the treatment of pain.

The term sub therapeutic dose is used to describe to describe a dose lower than that at which the component shows efficacy as a monotherapy.

Other advantages for the combination include the potential for oral dosing instead of intravenous or sub-cutaneous dosing. The combination may also result in a lower cost of treatment and provide a lower risk of immunogenicity.

Particularly suitable anti NGF antibodies include Tanezumab, Fasinumab, Fulranumab and MEDI735.

Particularly suitable anti-NGF antibodies are Tanezumab and Fasinumab.

In a preferred embodiment the anti NGF antibody is Tanezumab.

In another preferred embodiment the anti NGF antibody is Fasinumab.

In a particularly preferred embodiment, the present invention provides for the use of a SOS1 inhibitor in combination with a sub therapeutic dose of Tanezumab, for the treatment of pain. Optionally, both the SOS1 inhibitor and Tanezumab are administered at a sub therapeutic dose.

The term sub therapeutic dose is used to describe to describe a dose lower than that at which the component shows efficacy as a monotherapy.

Suitable SOS1 inhibitors for use in the present invention have an IC₅₀'s in the HTRF binding assay of less than or equal to 5 micromolar.

Particularly suitable SOS1 inhibitors have an IC₅₀ of less than 100 nanomolar.

In a particularly preferred embodiment the SOS1 inhibitors have an IC₅₀ of 1 nanomolar or less.

The SOS1 inhibitors of the present invention also show selectivity for SOS1 over additional targets. Suitably, the SOS1 inhibitors of the present invention show selectivity of greater than or equal to 100 fold over one or more of the following targets: MEK 1, MEK 2, TrkA kinase, TrkB kinase, TrkC kinase, C-Raf, B-Raf, PI3 kinase, AKT and ERK.

Further suitable SOS1 inhibitors for use in combination with anti NGF antibodies, particularly Tanezumab or Fusinimab are:

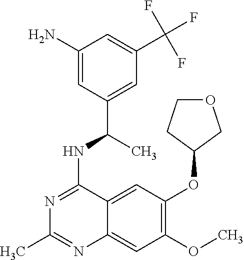

BI 3406 (N-[(1R)-1-[3-amino-5-(trifluoromethyl)phenyl]ethyl]-7-methoxy-2-methyl- 6[(3S)-oxolan-3-yl]oxyquinazolin-4-amine)

Synthesis of this Molecule is Published in WO2018115380

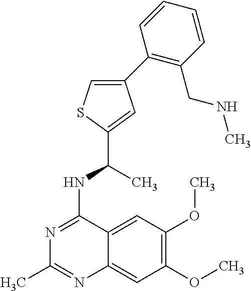

Bay 293 (6,7-dimethoxy-2-methyl-N-[(1R)-1-[4-[2-(methylaminomethyl)phenyl]thiopen-2-yl]ethyl]quinazolin-4-amine)

### Synthesis Published in PNAS 2019 116 (7) 2551-2560

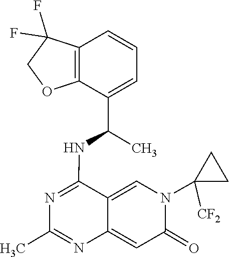

4-[[(1R)-1-(3,3-difluoro-2H-1-benzofuran-7-yl)ethyl]amino]-6-[1-(difluoromethyl)cyclopropyl]-2-methylpyrido[4,3-d]pyrimidin-7-one

### Synthesis Published in WO2019122129

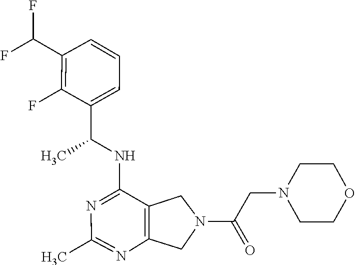 [SMILES: Cc1nc2c(c(N[C@H](C)c3cccc(C(F)F)c3F)n1)CN(C(=O)CN1CCOCC1)C2]

### Synthesis Published in WO2020180770

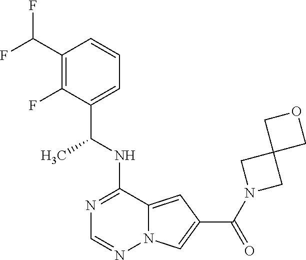 [SMILES: C[C@@H](Nc1ncnn2cc(C(=O)N3CC4(COC4)C3)cc12)c1cccc(C(F)F)c1F]

### Synthesis Published in WO2020180768.

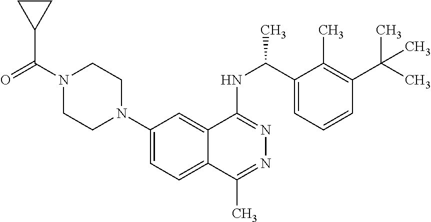 [SMILES: Cc1c([C@@H](C)Nc2nnc(C)c3ccc(N4CCN(C(=O)C5CC5)CC4)cc23)cccc1C(C)(C)C]

(R)-cyclopropyl(4-(1-methyl-4-((1-(2-methyl-3-(frifluromethyl)phenyl)ethyl)amino)phthalazine-6-yl)piperazin-1-yl)methanone

Synthesis published in WO2021127429

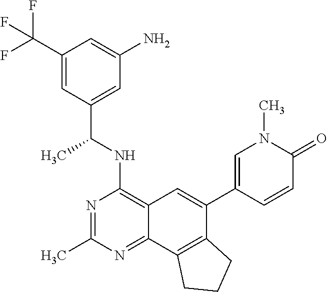 [SMILES: Cc1nc(N[C@H](C)c2cc(N)cc(C(F)(F)F)c2)c2cc(-c3ccc(=O)n(C)c3)c3c(c2n1)CCC3]

(R)-5-(4-((1-(3-amino-5-(trifluoromethyl)phenyl)ethyl)amino)-2-methyl-8,9-dihydro-7H-cyclopenta[h]quinazolin-6-yl)-1methylpyridin-2(1H)-one

### Synthesis Published in WO2021105960

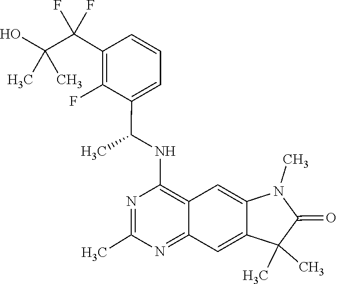 [SMILES: Cc1nc(N[C@H](C)c2cccc(C(F)(F)C(C)(C)O)c2F)c2cc3c(cc2n1)C(C)(C)C(=O)N3C]

(R)-4-((1-(3-(1,1-difluoro-2-hydroxy-2-methylpropyl)-2-fluorophenyl)ethyl)amino)-2,6,8,8-tetramethyl-6,8-dihyro-7H-pyrrolo[2,3-g]quinazolin-7-one

### Synthesis Published in WO2021130731

Particularly suitable SOS-1 inhibitors for use in combination with Tanazumab are BI 3406 (N-[(1R)-1-[3-amino-5-(trifluoromethyl)phenyl]ethyl]-7-methoxy-2-methyl-6-[(3S)-oxolan-3-yl]oxyquinazolin-4-amine) and Bay 293 (6,7-dimethoxy-2-methyl-N-[(1R)-1-[4-[2-(methylaminomethyl)phenyl]thiophen-2-yl]ethyl]quinazolin-4-amine)

Particularly suitable SOS-1 inhibitors for use in combination with Fasinumab are BI 3406 (N-[(1R)-1-[3-amino-5-(trifluoromethyl)phenyl]ethyl]-7-methoxy-2-methyl-6-[(3S)-oxolan-3-yl]oxyquinazolin-4-amine) and Bay 293 (6,7-dimethoxy-2-methyl-N-[(1R)-1-[4-[2-(methylaminomethyl)phenyl]thiophen-2-yl]ethyl]quinazolin-4-amine)

Suitable Ras inhibitors for use in the present invention have an IC₅₀'s in the binding assay of less than or equal to 5000 nM.

Particularly suitable Ras inhibitors have an IC₅₀ of less than 100 nanomolar.

In a particularly preferred embodiment the Ras inhibitors have an IC₅₀ of 1 nanomolar or less.

Suitable Ras Inhibitors include:

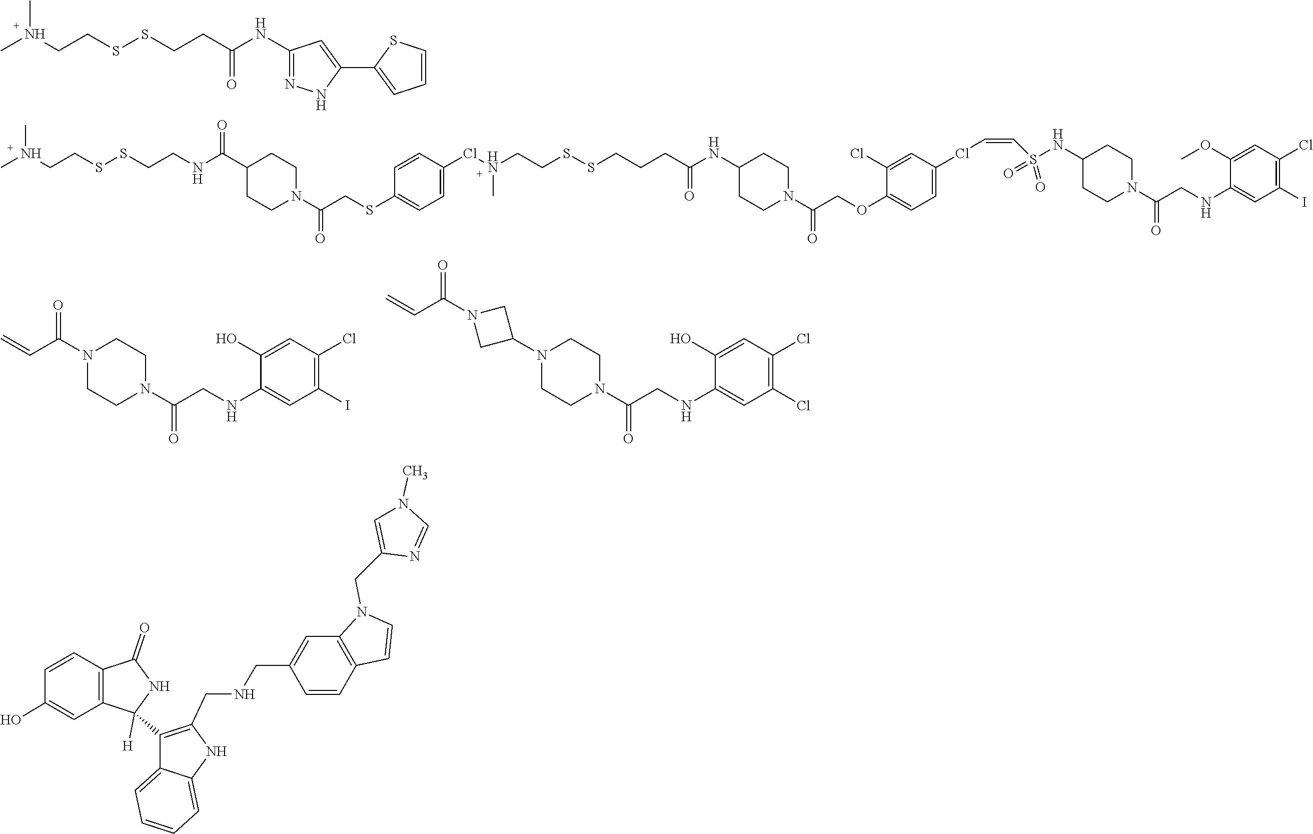

BI-2852

(3S)-5-hydroxy-3-[2-[[[1-[(1-methylimidazol-4-yl)methyl]indol-6-yl]methylamino]methyl]-1H-indol-3-yl]-2,3-dihydroisoindol-1-one

ARS-3248/JNJ74699157

ARS-1620

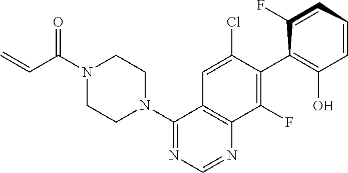

ARS-853

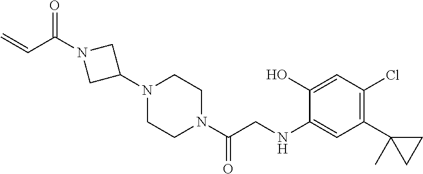 [SMILES: C=CC(=O)N1CC(N2CCN(C(=O)CNc3cc(C4(C)CC4)c(Cl)cc3O)CC2)C1]

AMG-510

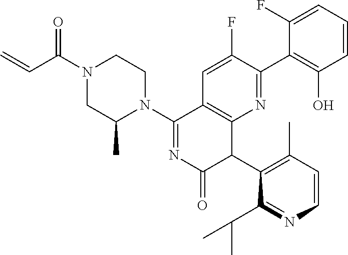

MRTX-849

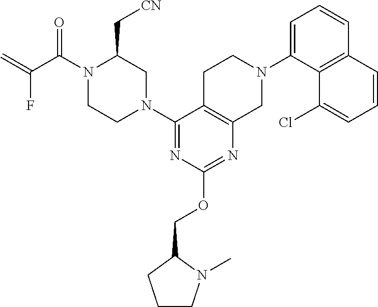

And BBP-454

Particularly suitable Ras inhibitors for use in combination with Fasinumab are BI 2852 (3S)-5-hydroxy-3-[2-[[[1-[(1-methylimidazol-4-yl)methyl]indol-6-yl]methylamino]methyl]-1H-indol-3-yl]-2,3-dihydroisoindol-1-one

Particularly suitable Ras inhibitors for use in combination with Tanazumab are BI 2852 (3S)-5-hydroxy-3-[2-[[[1-[(1-methylimidazol-4-yl)methyl]indol-6-yl]methylamino]methyl]-1H-indol-3-yl]-2,3-dihydroisoindol-1-one

Without being bound by theory, its believed that SOS1 inhibitors and Ras inhibitors act to treat pain in the following way:

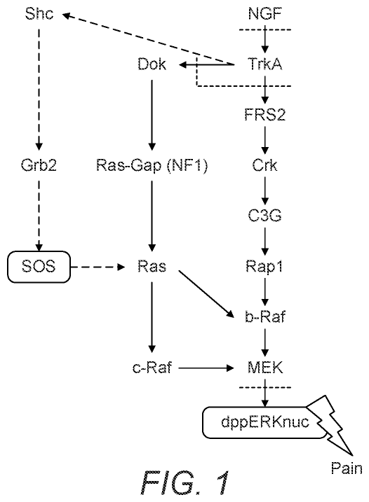

Nerve growth factor (NGF) is a protein that binds to the NGF receptor (TrkA), leading to the upregulation of genes involved in nociception. NGF is known to be an important contributor to the development of chronic pain. The NGF binding to TrkA and subsequent signal transduction culminates in the nuclear accumulation of diphopshorylated Extracellular signal-regulated kinase (dppERKnuc) in neurons, upregulating pain genes, as shown in FIG. 1.

Levels of SOS & molecules influenced by it downstream such as Ras feed into this cascade, with greater levels of SOS and RAS leading to higher levels of bRAF and MEK, leading to the accumulation of diphopshorylated Extracellular signal-regulated kinase (dppERKnuc), leading to higher levels of Pain. By inhibiting this pathway it is possible to control the formation of RAS GTP. By lowering levels of RAS GTP, pain is reduced.

The term pain includes but is not limited to: acute pain; chronic pain; inflammatory pain; nociceptive pain; neuropathic pain; hyperalgesia; allodynia; central pain; cancer pain; post-operative pain; visceral pain; musculo-skeletal pain; heart or vascular pain; head pain including migraine; orofacial pain, including dental pain; and back pain.

In more detail, suitable pain for treatment includes but is not limited to:
(a) acute pain and/or spontaneous pain,
(b) chronic pain and or on-going pain,
(c) inflammatory pain including any one of arthritic pain, pain resulting from osteoarthritis or rheumatoid arthritis, resulting from inflammatory bowel diseases, psoriasis and eczema
(d) nociceptive pain,
(e) neuropathic pain, including painful diabetic neuropathy or pain associated with post-herpetic neuralgia,
(f) hyperalgesia,
(g) allodynia,
(h) central pain, central post-stroke pain, pain resulting from multiple sclerosis, pain resulting from spinal cord injury, or pain resulting from Parkinson's disease or epilepsy,
(i) cancer pain,
(j) post-operative pain,
(k) visceral pain, including digestive visceral pain and non-digestive visceral pain, pain due to gastrointestinal (GI) disorders, pain resulting from functional bowel disorders (FBD), pain resulting from inflammatory bowel diseases (IBD), pain resulting from dysmenorrhea, pelvic pain, cystitis, interstitial cystitis or pancreatitis,
(l) musculo-skeletal pain, myalgia, fibromyalgia, spondylitis, sero-negative (non-rheumatoid) arthropathies, non-articular rheumatism, dystrophinopathy, Glycogenolysis, polymyositis, pyomyositis,
(m) heart or vascular pain, pain due to angina, myocardical infarction, mitral stenosis, pericarditis, Raynaud's phenomenon, scleredoma, scleredoma or skeletal muscle ischemia,
(n) head pain including migraine, migraine with aura, migraine without aura cluster headache, tension-type headache.
(o) orofacial pain, including dental pain, temporomandibular myofascial pain or tinnitus, or
(p) back pain, bursitis, menstrual pain, migraine, referred pain, trigeminal neuralgia, hypersensitisation, pain resulting from spinal trauma and/or degeneration or stroke.

Treatment of pain includes, but is not limited to, preventing, ameliorating, controlling, reducing incidence of, or delaying the development or progression of pain.

Particularly suitable pain indications include Osteoarthritis and cancer pain.

In another embodiment a suitable indication is osteoarthritis.

According to another aspect of the invention there is provided the compounds of the present invention for separate, sequential or simultaneous use in a combination combined with a second pharmacologically active compound. Preferably the second pharmacologically active compound of the combination may include but is not limited to;
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6- 13-dione (TAK-637), 5-[[(2R,3S)-2-[(1R)-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′; 6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]-pyrimidin-7-one, 5-(5-acetyl-2-propoxy-3-pyridinyl)-3-ethyl-2-(1-isopropyl-3-azetidinyl)-2,6-dihydro-7H-pyrazolo[4,3d]-pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]- 4-methylbenzenesulfonamide or 4-[(1 S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine; or
a 5-HT3 antagonist, such as ondansetron;
   and the pharmaceutically acceptable salts and solvates thereof.

The invention further provides a pharmaceutical formulation comprising a compound of formula I, as defined above, or a pharmaceutically acceptable salt or solvate thereof, and a pharmaceutically acceptable adjuvant, diluent or carrier. The pharmaceutical formulation may further comprise one or more additional active agents for the treatment of a disorder mentioned above.

The invention further provides a pharmaceutical kit comprising a compound of formula I, as defined above, or a pharmaceutically acceptable salt or solvate thereof, and one or more additional active agents, as a combined preparation for separate, simultaneous or sequential administration in the treatment of a disorder mentioned above.

The invention further provides a method of treatment of a disorder mentioned above in a mammal (especially a human), comprising administration of a therapeutically effective amount of a compound of formula I, as defined above, or a pharmaceutically acceptable salt or solvate thereof, to a mammal in need of such treatment.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of the invention may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(d/-lactic-coglycolic)acid (PGLA) microspheres.

### Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as /-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 to 10,000 μg of the compound of the invention. The overall daily dose will typically be in the range 1 μg to 10 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula I in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### Dosage

For administration to human patients, the total daily dose of the compounds of the invention is typically in the range 0.5 mg to 3000 mg depending, of course, on the mode of administration. For example, oral administration may require a total daily dose of from 3 mg to 3000 mg, while an intravenous dose may only require from 0.5 mg to 500 mg. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

### Experimental

### Materials and Methods.

### Identification of SOS1 Inhibitors:

The following method is based on the HTRF binding assay described in Hillig et al, PNAS February 12, 2019, vol. 116, no. 7, 2551-2560

Measurement and evaluation of inhibition data, calculation of IC₅₀ values.

Homogeneous time-resolved fluorescence (HTRF) was measured with a PHERAstar reader (BMG, Germany) using the HTRF module (excitation: 337 nm; emission 1: 620 nm, emission 2: 665 nm). The ratio of the emissions at 665 and 620 nm was used as the specific signal for further evaluation. The data were normalized using the controls: DMSO=0% inhibition, inhibition control wells with inhibitor control solution=100% inhibition. Compounds were tested in duplicates at up to 11 concentrations (e.g. 20 μM, 5.7 μM, 1.6 μM, 0.47 μM, 0.13 μM, 38 nM, 11 nM, 3.1 nM, 0.89 nM, 0.25 nM and 0.073 nM). IC50 values were calculated using a four-parameter fit, with a commercial software package (Genedata Screener, Switzerland). KRASG12C activation by SOS1cat assay (“On-assay”). This assay quantifies SOS1cat mediated loading of KRASG12C—GDP with a fluorescent GTP analogue. Detection of successful loading was achieved by measuring resonance energy transfer from anti-GST-terbium (FRET donor) bound to GST-KRASG12C to the loaded fluorescent GTP analogue (FRET acceptor). The fluorescent GTP analogue EDA—GTP—DY-647P1 [2′/3′-O-(2-aminoethyl-carbamoyl)guanosine-5′-triphosphate labelled with DY-647P1 (Dyomics GmbH, Germany)] was synthesized by Jena Bioscience (Germany) and supplied as a 1 mM aqueous solution. A KRASG12C working solution was prepared in assay buffer [10 mM HEPES pH 7.4 (AppliChem), 150 mM NaCl (Sigma), 5 mM MgCl2 (Sigma), 1 mM DTT (Thermo Fisher), 0.05% BSA Fraction V pH 7.0 (ICN Biomedicals), 0.0025% (v/v) Igepal (Sigma)] containing 100 nM GST-KRASG12C and 2 nM anti-GST-terbium (Cisbio, France). A SOS1cat working solution was prepared in assay buffer containing 20 nM SOS1cat and 200 nM EDA—GTP—DY-647P1. An inhibitor control solution was prepared in assay buffer containing 200 nM EDA-GTP—DY-647P1 without SOS1cat. All steps of the assay were performed at 20° C. A volume of 2.5 μL of the KRASG12C working solution was added to all wells of the test plate using a Multidrop dispenser (Thermo LabSystems). After 10 min, 2.5 μL of the SOS1cat working solution was added to all wells, except for the inhibitor control solution wells. After 30 min incubation, HTRF was measured.

### Identification of Ras Inhibitors

Ras inhibitors were identified using the following assay from Kessler et al, Proc Natl Acad Sci USA. 2019 Aug 6; 116(32): 15823-15829

Alpha Screen Assays 220; an in vitro cell free method to measure SOS:RAS interactions.

Measurements of various protein—protein interactions were performed using the Alpha 221 Screen technology developed by Perkin Elmer. Recombinant RAS proteins (H-, N-, K-222 RAS variants; all KRAS variants are based on KRAS isoform 4B (uniprot id P01116-2); 223

KRAS (G12D) 1-169, N-terminal 6His-tag, C-terminal avi-tag was from Xtal 224 BioStructures, Inc., KRAS (G12C) 1-169, C-terminal avi-tag, biotinylated, mutations: 225 C51S, C80L, C118S, NRAS(wt) 1-172, C-terminal avi-tag, biotinylated; HRAS(wt) 1-226 166, C-terminal avi-tag, biotinylated). Biotinylation was performed in vitro with 227 recombinant BirA biotin-protein ligase as recommended by the manufacturer (Avidity 228 LLC, Aurora, Colorado, USA). Interacting proteins such as SOS1 (564-1049, N-terminal 229 GST-tag, TEV cleavage site), cRaf (1-303, N-terminal GST-tag, TEV cleavage site) and 230

PI3KA-RBD (160-317, N-term-HIS_GST-tag) were expressed as glutathione S 231 transferase (GST) fusions. Accordingly, the Alpha Screen beads were glutathione coated 232 Alpha Lisa acceptor beads (Perkin Elmer AL 109 R) and Alpha Screen Streptavidin 233 conjugated donor beads (Perkin Elmer 6760002L). Nucleotides were purchased from 234 Sigma (GTP #G8877, GDP #G7127), Tween-20 from Biorad (#161-0781). All 235 interaction assays were carried out in PBS, containing 0.1% bovine serum albumin, 236 0.05% Tween-20 and 10 μM of the corresponding nucleotide. Assays were carried out in 237 white ProxiPlate-384 Plus plates (Perkin Elmer #6008280) in a final volume of 20 μL. In 238 brief, biotinylated RAS proteins (10 nM final concentration) and GST-SOS1, GST-PI3K 239 or GST-CRAF (10 nM final) were mixed with glutathione acceptor beads (5 μg/mL final 240 concentration) in buffer, containing the corresponding nucleotides (GDP or GTP for 241 assays containing SOS1, only GTP for interaction assays containing PI3K or CRAF) and 242 were incubated for 30 min at room temperature. After addition of streptavidin donor 243 beads (5 μg/mL final concentration) under green light, the mixture was further incubated 244 for 60 min in the dark at room temperature. Single oxygen induced fluorescence was 245 measured at an Enspire multimode plate reader (Perkin Elmer) according to the 246 manufacturer's recommendations. Data were analyzed using the Graph Pad Prism data 247 software.

The ability of SOS1 inhibitors and Ras inhibitors to treat pain was measured using the assay below, based on the NGF stimulated PC12 assay (Sasagawa et al, NATURE CELL BIOLOGY VOLUME 7, NUMBER 4, APRIL 2005, 365-373).

Inhibition of Nerve Growth Factor (NGF) stimulated phospho-Extracellular Regulated Kinase 1 and 2 (pERK1/2) activation in the PC-12 cell line by 813406.

Homogeneous Time-Resolved Fluorescence (HTRF) assay:

### BI-3406 Inhibition at Different NGF Stimulus Concentrations:

The inhibitory effect of a selective, small molecule Rat sarcoma:Son of Sevenless1 (RAS:SOS1) inhibitor, BI-3406 (Selleck Chemicals), was monitored via an HTRF readout measuring phosphorylation of ERK1/2 following NGF activation. All assays were performed in rat adrenal PC-12 cells (Merck) that had been serum-starved for a period of 24 hours in RPMI-1640 growth medium (Gibco) supplemented with 1% heat-inactivated horse serum (Merck), 0.5% heat-inactivated fetal bovine serum (FBS), 1% penicillin-streptomycin and 2 mM L-Glutamine, unless specified otherwise. Reagents from the HTRF commercial kit (Cisbio) were prepared according to the manufacturer's instructions.

### Assay Procedure:

PC-12 cells were isolated from routine cellular culture and plated at an appropriate cell density in 384-well plates (typically 25,000 cells per well) for 24 hours under serum-starved conditions. Following incubation, PC-12 cells were pre-treated with working concentrations of BI-3406 across an appropriate concentration response range for 30 minutes (37° C./5% CO₂). Duplicate concentration response curves for BI-3406 were set-up per NGF (Merck) concentration tested. Following the 30-minute compound pre-incubation, PC-12 cells were treated with NGF (titrations of NGF from 250 ng/mL to 10 ng/mL were tested), and subsequently incubated for a 5-minute period (37° C./5% CO₂). Following 5 minutes of NGF treatment, lysis buffer from the commercial HTRF kit was applied to the PC-12 cells for 30-minutes of incubation with shaking (20° C. at 600rpm). An appropriate volume of lysate was harvested per well and transferred to a separate 384-well Proxiplate (Perkin Elmer) to which a 5× concentrated HTRF kit antibody mix was dispensed into each lysate sample well. A 2-hour incubation at room temperature was performed prior to fluorescence signal determination using a microplate reader (PHERAstar FSX, BMG Labtech).

### Data

| | | Tanezumab | BI3406 | BI3406 | BI3406 |
|---|---|---|---|---|---|
| TIME(APPROX) | Vehicle | 3 mg/kg | 25 mg/kg | 50 mg/kg | 100 mg/kg |
| BASELINE | 99.85 | 102.78 | 100.36 | 101.04 | 98.92 |
| CFA | 57.69 | 58.95 | 56.47 | 57.89 | 55.8 |
| DRUG TREATED | 68.89 | 82.54 | 68.18 | 71.76 | 66.96 |
| 0.5 h | | | | | |
| DRUG TREATED | 63.01 | 94.11 | 69.64 | 77.59 | 81.75 |
| 8.5 h | | | | | |
| DRUG TREATED | 60.52 | 89.88 | 67.26 | 78.09 | 91.15 |
| 24.5 h | | | | | |
| DRUG TREATED | 61.05 | 84.25 | 65.06 | 74.11 | 84.56 |
| 32.5 h | | | | | |

| | SEM | SEM | DRUG 25 | DRUG 50 | DRUG 100 |
|---|---|---|---|---|---|
| | BASE | tanezumab | mg/kg | mg/kg | mg/kg |
| Base | 1.4 | 2 | 1.42 | 2.1 | 2.03 |
| CFA | 2.05 | 1.75 | 2.96 | 2.63 | 2.03 |
| Drog 0.5 | 3.71 | 4.46 | 2.77 | 4.78 | 5.22 |
| Drug 8.5 | 2.27 | 4.02 | 4.57 | 4.02 | 5.48 |
| Drug 24.5 | 2.59 | 4.11 | 2.98 | 4.83 | 5.41 |
| Drug32.5 | 1.98 | 3.33 | 2.66 | 3.69 | 4.8 |
| Base | 1.4 | 2 | 1.42 | 2.1 | 2.03 |
| CFA | 2.05 | 1.75 | 2.96 | 2.63 | 2.03 |
| Drog 0.5 | 3.71 | 4.46 | 2.77 | 4.78 | 5.22 |
| Drug 8.5 | 2.27 | 4.02 | 4.57 | 4.02 | 5.48 |
| Drug 24.5 | 2.59 | 4.11 | 2.98 | 4.83 | 5.41 |
| Drug32.5 | 1.98 | 3.33 | 2.66 | 3.69 | 4.8 |

### Analysis.

i. BI-3406 IC₅₀ plot against NGF activation:

Percentage inhibition values were calculated per compound concentration by normalising the sample data to the high and low controls used within each plate (+/−25 ng/mLNGF respectively). Percentage inhibition values from each duplicate BI-3406 concentration response curve were meaned to provide a data point per concentration. Mean percentage inhibition values were then used to determine the 50% inhibitory concentration (IC₅₀) and maximum % efficacy (inhibition) was (maximum % efficacy BI3406/ maximum % efficacy given by −NGF versus+NGF difference), using a four-parameter logistic fit where y=A xnH/(EC50nH+xnH), and x and y represent the test agent concentration and % cell pERK signal, respectively. The parameter EC50 is test agent concentration half-maximal output and A is the maximal inhibition (efficacy), while nH is the Hill coefficient (GraphPad Prism). Response data were then plotted against the molar logarithm for each BI-3406 compound concentration together with the determined fit results for display purposes. Error bars represent one standard deviation.
ii. BI-3406 IC₅₀ versus NGF concentration:

IC₅₀ values were calculated for BI-3406 at NGF concentrations, 250 ng/mL, 200 ng/mL, 150 ng/mL, 100 ng/mL, 50 ng/mL, 25 ng/mL and 10 ng/mL following the analysis described in section ‘i’. The resulting geometric mean of the IC₅₀ values (y-axis) per NGF concentration (x-axis) were subsequently plotted within a separate graph. Error bars represent one standard deviation calculated from 2-8 separate IC50 replicates.
iii. Percentage Maximum Efficacy versus NGF concentration:

Percentage inhibition values were calculated per compound concentration across an appropriate BI-3406 concentration response range as described in section T. The mean percentage inhibition value calculated for the top concentration of BI-3406 compound tested per NGF concentration was calculated from 2-8 replicates and subsequently plotted within a separate graph to show percentage maximum efficacy (y-axis) against NGF concentration (x-axis). Error bars represent one standard deviation.

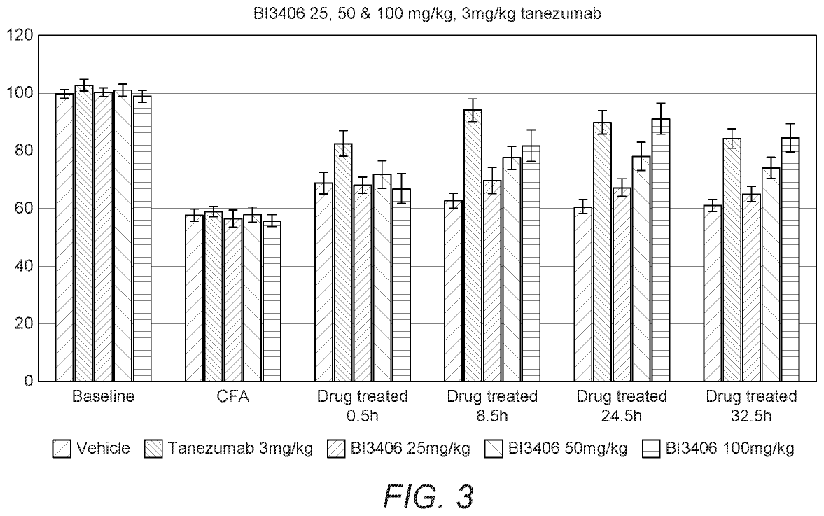

The results are expressed graphically in FIG. 3

Combination work; co-operativity of antiNGF and BI-3406 inhibition.

The combined inhibitory effect of a selective, small molecule RAS:SOS inhibitor, BI-3406 (Selleck Chemicals), and known antibody based NGF inhibitor, Anti-NGF was monitored via an HTRF readout measuring phosphorylation of ERK1/2 by NGF activation. All assays were performed in rat adrenal PC-12 cells (Merck) that had been serum-starved for a period of 24 hours in RPMI-1640 growth medium (Gibco) supplemented with 1% heat-inactivated horse serum (Merck), 0.5% heat-inactivated fetal bovine serum (FBS), 1% penicillin-streptomycin and 2 mM L-Glutamine unless specified otherwise. Reagents from the HTRF commercial kit (Cisbio) were prepared according to the manufacturer's instructions.

### Assay Procedure:

PC-12 cells were isolated from routine cellular culture and plated at an appropriate cell density (typically 25,000 cells per well) in 384-well plates for 24 hours under serum-starved conditions. Working preparations of varying Anti-NGF (Abcam) concentrations and a fixed NGF concentration were prepared in serum-starved media and pre-incubated for 30 minutes (37° C./5% CO₂). Simultaneously, PC-12 cells were pre-treated with working concentrations of BI-3406 for 30 minutes (37° C./5% CO₂). Duplicate concentration response curves for BI-3406 were set-up per Anti-NGF; NGF combination tested. Following 30 minutes of pre-incubation, PC-12 cells were treated with an appropriate Anti-NGF; NGF combination (titrations of Anti-NGF ranging from 30 μg/mL to 0 μg/mL against a fixed 250 ng/mL concentration of NGF were tested), and subsequently incubated for a 5-minute period (37° 0/5% CO₂). Following 5 minutes of Anti-NGF; NGF treatment, lysis buffer from the commercial HTRF kit was applied to the PC-12 cells for 30-minutes of incubation with shaking (20° C. at 600 rpm). An appropriate volume of lysate was harvested per well and transferred to a separate 384-well Proxiplate (Perkin Elmer) to which a 5× concentrated HTRF kit antibody mix was dispensed into each lysate sample well. A 2-hour incubation at room temperature was performed prior to fluorescence signal being measured in a microplate reader (PHERAstar FSX, BMG Labtech).

### Analysis.

i. BI-3406 percentage efficacy plot against Anti-NGF;NGF challenge:

Percentage inhibition values were calculated per compound concentration by normalising the sample data to the high and low controls used within each plate (+/−250 ng/mL NGF respectively). Percentage inhibition values from each duplicate BI-3406 concentration response curve were meaned to provide a data point per concentration. Mean percentage inhibition values were then used to determine the 50% inhibitory concentration (IC₅₀) and maximum inhibition (efficacy) (maximum % inhibition (efficacy) BI3406/maximum % inhibition (efficacy) given by—NGF) and using a four-parameter logistic fit where y=A xnH/(EC50nH+xnH), where x and y represent the test agent concentration and % cell pERK signal, respectively. The parameter EC50 is test agent concentration half-maximal effect and A is the maximal output (efficacy), while nH is the Hill coefficient (GraphPad Prism). Response data were then plotted against the molar logarithm for each BI-3406 compound concentration together with the determined fit results for display purposes. Error bars represent one standard deviation. The mean percentage inhibition value calculated for both the top and bottom concentration of BI-3406 compound tested per Anti-NGF:NGF combination was extracted and compared in tabular form across the different Anti-NGF:NGF combinations tested.

### Western Blot:

### NGF Challenge Against BI-3406 Inhibition:

The inhibitory effect of a selective, small molecule RAS:SOS inhibitor, BI-3406 (Selleck Chemicals), was monitored via a Western Blot based readout (Jess, Protein Simple) measuring phosphorylation of ERK1/2 by NGF activation. All assays were performed in rat adrenal PC-12 cells (Merck) that had been serum-starved for a period of 24 hours in RPM1-1640 growth medium (Gibco) supplemented with 1% heat-inactivated horse serum (Merck), 0.5% heat-inactivated fetal bovine serum (FBS), 1% penicillin-streptomycin and 2 mM L-Glutamine unless specified otherwise. Reagents from the Jess Separation Module commercial kit (Protein Simple) were prepared according to the manufacturer's instructions.

### Assay Procedure:

PC-12 cells were isolated from routine cellular culture and plated at an appropriate cell density in 6-well plates for 24 hours under serum-starved conditions. Following incubation, PC-12 cells were pre-treated with working concentrations of BI-3406 for 30 minutes (37° C./5% CO₂). Duplicate concentration response curves for BI-3406 were set-up per NGF (Merck) concentration tested. Following the 30-minute compound pre-incubation, PC-12 cells were treated with an appropriate concentration of NGF, and subsequently incubated for a 5-minute period (37° C./5% CO₂). Following 5 minutes of NGF treatment, PC-12 cellular suspensions were transferred to falcon tubes and centrifuged (300×g for 5 minutes at 4° C.). Cellular supernatant was discarded, and the remaining cell pellet was washed with of ice-cold PBS. This was repeated for 3×PBS washes, before ice-cold lysis buffer containing protease and phosphatase inhibitors was added to the dry cell pellet. Tubes were subsequently sonicated for 30-seconds and stored on ice for 15-minutes with regular inversion to ensure completion of cellular lysis. Following lysis, samples were centrifuged (13,000×g for 5 minutes at 4° C.), and supernatant extracted to be prepared for Western Blot analysis. Supernatant samples were prepared for loading into Jess capillary cassettes according to the manufacturer's instructions. Duplicate samples per BI-3406 concentration tested were loaded within the same run. Data were analysed as described in i.

### Cell Culture and Growth-Factor Treatments.

PC12 cells (1) were purchased from the American Type Culture Collection (Rockville, MD) and cultured in RPMI-1640 (Biowhittaker, Walkersville, MD) with 10% horse serum (Life Technologies, Grand Island, NY) and 5% fetal bovine serum (Hyclone, Logan, UT). Cell viability was assessed by trypan blue dye exclusion. Prior to assays, cells were starved in DMEM for 16 h, then stimulated with Nerve Growth Factor (NGF-β; mouse submaxillary glands, Sigma, St. Louis, MO) which was dissolved in RPMI-1640 at the concentration of 20 ng/μl and then diluted to the appropriate concentration before use.

### Immunofluorescence Assays.

Cells were plated on poly-L-lysine-coated coverslips or gridded glass-bottomed dishes, serum starved and stimulated with the indicated concentrations of NGF. Cells were fixed with 4% paraformaldehyde for 10 min at room temperature, permeabilized in 0.2% Triton X-100 for 10 min at room temperature or 100% methanol for 10 min at −20° C. and then blocked with 1% BSA for 30 min at room temperature. Cells were then incubated with primary antibodies anti-phospho-ERK1/2 (1:200) antibody for 1-2 h at RT, followed by secondary antibody (Alexa Fluor 647 anti-mouse IgG (1:500)) antibodies for 1 h at room temperature. All antibodies were supplied by Cell Signalling Technology, Danvers, MA, USA. Following equilibration, cells were then washed and the Fluorescence was measured using a spectrofluorometer (Molecular Devices Spectramax, San Jose, CA 95134 USA).

### Test Compounds.

Anti NGF monoclonal antibodies were supplied by Abcam (Cambridge, MA, USA). BI-3406 was supplied by Medchem Express, NJ, USA.

### Data Analysis.

Data were analysed using Graph Pad Prism.

### Results

A number of structurally diverse SOS1 inhibitors including BI 3406 were tested in the Inhibition of Nerve Growth Factor (NGF) stimulated phospho-Extracellular Regulated Kinase 1 and 2 (pERK1/2) activation in the PC-12 cell line assay against a suitable control. All the tested compounds have an IC₅₀ in SOS1 assay below 5.

All showed a level of efficacy between 40-70%.

BI-3406 had an IC₅₀ of 10-20 nM

A Ras inhibitor was tested in the same assay. It showed a level of efficacy between 40-70%.

The ability of an NGF inhibitor Tanezumab in combination with a SOS1 inhibitor BI-3406 to treat pain was measured in the Inhibition of Nerve Growth Factor (NGF) stimulated phospho-Extracellular Regulated Kinase 1 and 2 (pERK1/2) activation in the PC-12 cell line assay.

At a concentration of 200 nM, tanezumab had 100% efficacy

At a concentration of 50 nM, Tanezumab had 90% efficacy.

BI-3406 at a concentration of 500 nM had 40% efficacy

Tanezumab at a concentration of 50 nM, in combination with BI-3406 at a concentration of 500 nM had ˜96% efficacy

The ability of an NGF inhibitor Tanezumab in combination with a Ras inhibitor to treat pain was also measured in the assay.

At a concentration of 200 nM, tanezumab had 100% efficacy

At a concentration of 50 nM, Tanezumab had 90% efficacy.

BI-3406 at a concentration of 500 nM had 40% efficacy

Tanezumab at a concentration of 50 nM, in combination with a Ras inhibitor had ˜97% efficacy.

Title: Investigation of BI3406 at 25 mg/kg in combination with a single dose of Tanezumab on the CFA-(Complete Freunds Adjuvant) induced hypersensitivity using weight-bearing in the C57BI6 mouse.

Aim of Study: To investigate the effects of BI3406 at 25 mg/kg (10 mL/kg, p.o., am and pm dosing, 2 days), in combination with a single dose of Tanezumab at 0.1, 0.3 and 1 mg/kg (10 mL/kg, i.p., T=0) and Tanezumab alone at 3 mg/kg (10 mL/kg, i.p., T=0) using weight-bearing (WB) assessments at 1 h post-dosing for BI3406 and +1 h, +9 h, +25 h and +33 h post Tanezumab administration.

Animals: n=50 male C57/BL6 mice 20.1 g-26.0 g (Charles River Order #4650568).

Environmental conditions: Animals were housed in groups of 5, in standard caging with free access to food and water on a 12 h/12 h light/dark cycle (lights on at 7:00 am).

Test compounds:
BI3406 at 25 mg/kg (10 mL/kg, i.p., BID, 2 days)
Tanezumab at 0.1, 0.3, 1 & 3 mg/kg (10 mL/kg, i.p., T=0)

Control group: Vehicle, 0.5% Hydroxy methylcellulose (10 mL/kg, p.o., BID, 2 days)

### Study Schedule:

On 3 days before CFA dose: habituation to WB apparatus, baseline readings are taken once.

24 h before dosing: intraplantar injection of 20 μl of CFA 1.5 mg/ml to the left hind paw.

1 h before dosing: post-CFA readings are taken for WB.

### On Day 1: Administration of Compounds

Tanezumab at 0.1, 0.3, 1 & 3 mg/kg (10 mL/kg, i.p.).
BI3406 at 25 mg/kg (10 mL/kg, i.p., BID, 2 days).

On days 1 and 2, weight-bearing measurements were taken at 1 h after each dose of BI3406 (see schedule above).

Day 3 animals were dosed with BI3406 and 3 animals per group were culled at each timepoint for collection of blood. Time points were 1 h, 2 h and 8 h after the last dose of BI3406. Animals were culled by a Schedule 1 method, blood was collected via cardiac puncture into EDTA tubes and centrifuged at 4° C. for 10 min, at 1500×g. The resulting plasma was removed and split into 2 aliquots. Samples were stored at −80° C. until shipment.

### Evaluation of Study: Weight-Bearing

Naive animals distribute their body weight equally between the two hind paws. However, when a painful insult is given to one hindpaw (i.e., sciatic nerve constriction or CFA intraplantar injection), the weight is re-distributed so that less weight is put on the affected paw (with a decrease in weight bearing on the injured paw). Animals were placed in the incapacitance tester (Linton Instruments, UK) with the hind paws on separate sensors. The average forces exerted by the left and the right hindlimb were recorded over 2 s. Weight-bearing readings were taken for both left and right hind paws. The ratio ipsilateral/contralateral was calculated and expressed in % (mean±s.e.m.).

### Results

| | | | BI3406 | BI3406 | BI3406 | |
|---|---|---|---|---|---|---|
| | | | 25 mg/kg + | 25 mg/kg + | 25 mg/kg + | |
| | | Tanezumab | 1 mg/kg | 0.3 mg/kg | 0.1 mg/kg | 25 mg/kg |
| TIME(APPROX) | Vehicle | 3 mg/kg | tanezumab | tanezumab | tanezumab | BI3406 |
| BASELINE | 103.56 | 97.99 | 106.54 | 102.55 | 102.5 | 100.36 |
| CFA | 62.3 | 58.57 | 62.64 | 62.12 | 62.06 | 56.47 |
| DRUG | 62.69 | 99.84 | 98.01 | 83.71 | 69.65 | 65.06 |
| TREATED | | | | | | |

| SEM | SEM | SEM |
|---|---|---|
| BASE | CFA | d2 PM |
| 3.16 | 1.8 | 2.72 |
| 2.87 | 2.34 | 4.54 |
| 3.01 | 1.96 | 2.87 |
| 1.97 | 2.99 | 3.86 |
| 3.18 | 2.29 | 3.96 |
| 1.42 | 2.96 | 2.66 |
| 3.16 | 1.8 | 2.72 |
| 2.87 | 2.34 | 4.54 |
| 3.01 | 1.96 | 2.87 |
| 1.97 | 2.99 | 3.86 |
| 3.18 | 2.29 | 3.96 |
| 1.42 | 2.96 | 2.66 |

Statistics: Two-Way RM Mixed model (treatment×repeated factor: time) (InVivoStat v3.7.0.0)

*p<0.05, **p<0.01 and ***p<0.001 significant difference when compared to the vehicle group at the same timepoint (n=9-10 per group). ###p<0.001 significant difference when compared to baseline within the same drug group.

### Analysis

A sub-analgesic dose of SOSi (BI3406, 25 mg/kg po) had no limited analgesic effect on its own but when combined with the NGF monoclonal antibody (Tanezumab), an enhanced analgesic response was observed. The enhanced analgesic effect of Tanezumab, when combined with a SOSi (BI3406, 25 mg/kg po), was dose related. A statistically significant enhancement of the analgesic response was observed with 0.3 mg/kg and 1.0 mg/kg Tanezumab when combined with SOSi (BI3406, 25 mg/kg). Combination studies produced analgesic effects that were comparable to higher doses of Tanezumab when dosed alone.

1. A method of treatment of pain, comprising the administration of an SOS1 inhibitor, or a pharmaceutically acceptable salt thereof, and/or a Ras inhibitor, or a pharmaceutically acceptable salt thereof, to a subject in need thereof.

2. The method of claim 1 wherein the SOS1 inhibitor, or a pharmaceutically acceptable salt thereof, has an IC₅₀ of less than or equal to 5 micromolar.

3. The method of claim 1 wherein the SOS1 inhibitor, or a pharmaceutically acceptable salt thereof, has an IC₅₀ of less than 100 nanomolar,

4. The method of claim 1 wherein the SOS1 inhibitor, or a pharmaceutically acceptable salt thereof, has an IC₅₀ of 1 nanomolar or less.

5. The method of claim 1 wherein the Ras inhibitor, or a pharmaceutically acceptable salt thereof, has an IC₅₀ of less than or equal to 500 nanomolar.

6. The method of claim 1 wherein the Ras inhibitor, or a pharmaceutically acceptable salt thereof, has an IC₅₀ of less than 100 nanomolar,

7. The method of claim 1 wherein the Ras inhibitor, or a pharmaceutically acceptable salt thereof, has an IC₅₀ of 1 nanomolar or less.

8. The method of claim 1 wherein the SOS1 inhibitor, or a pharmaceutically acceptable salt thereof, and/or Ras inhibitor, or a pharmaceutically acceptable salt thereof, shows selectivity of greater than or equal to 100 fold over one or more of the following targets: MEK 1, MEK 2, TrkA kinase, TrkB kinase, TrkC kinase, C-Raf, B-Raf, PI3 kinase, AKT and ERK.

9. The method of according to claim 1 wherein the SOS1 inhibitor is selected from 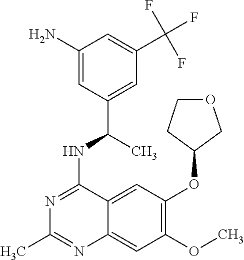
BI 3406 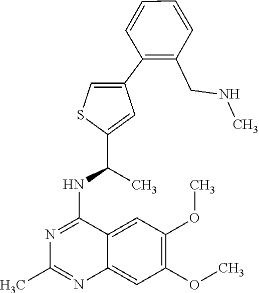
Bay 293 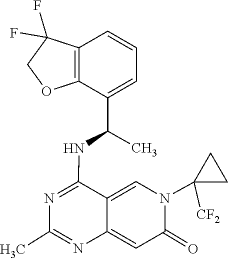
4-[[(1R)-1-(3,3-difluoro-2H-1-benzofuran-7-yl)ethyl]amino]-6-[1-(difluoromethyl)cyclopropyl]-2-methylpyrido[4,3-d]pyrimidin-7-one 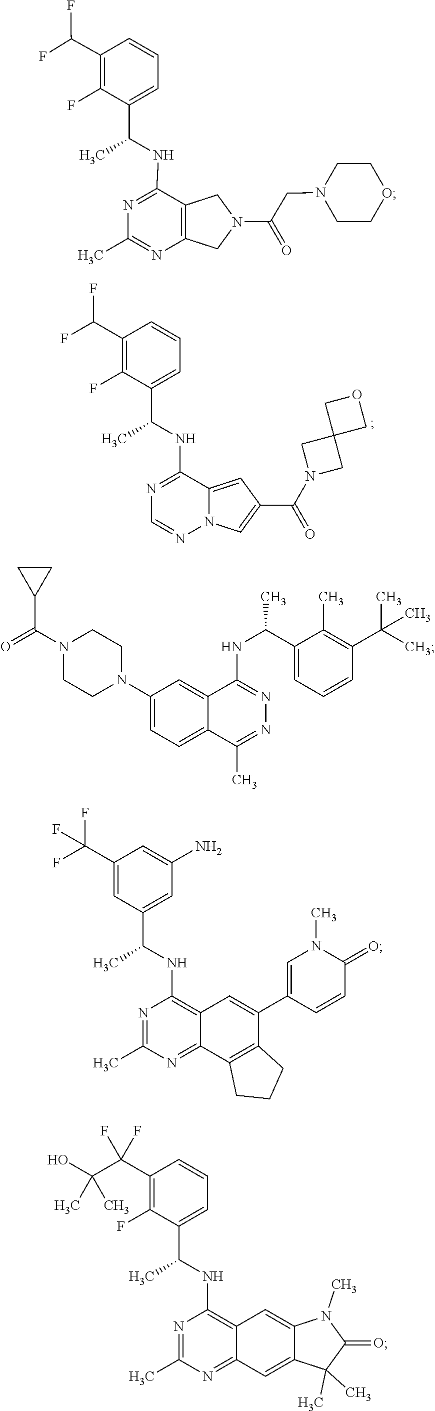
and BI-170963, or a pharmaceutically acceptable salt thereof.

10. The method according to claim 1, wherein the SOS1 inhibitor is selected from (BI-3406) & (BAY-293), or a pharmaceutically acceptable salt thereof.

11. The method according to claim 1 wherein the Ras inhibitor is selected from: 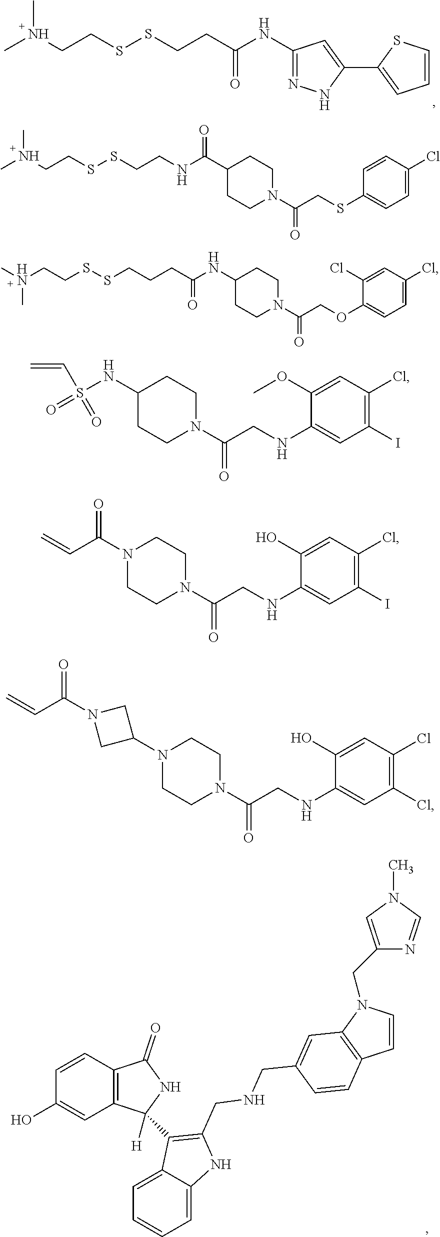 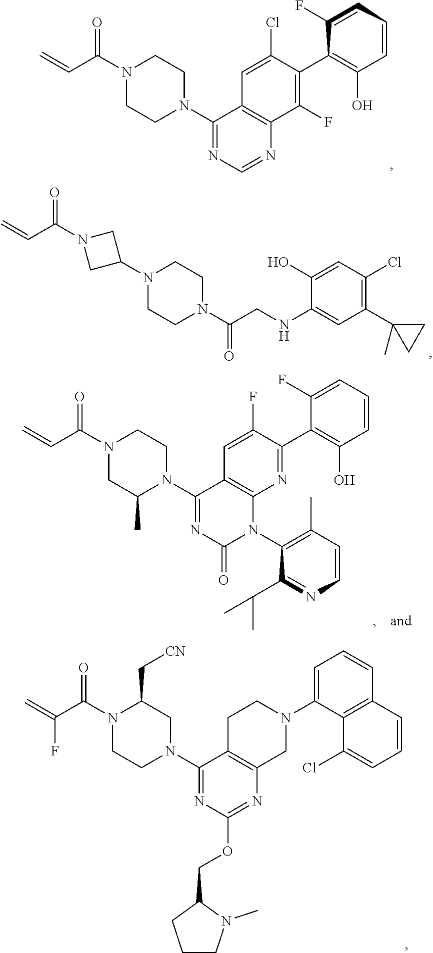
or a pharmaceutically acceptable salt thereof.

12. The method according to claim 1 wherein the Ras inhibitor is (3S)-5-hydroxy-3-[2-[[[1-[(1-methylimidazol-4-yl)methyl]indol-6-yl]methylamino]methyl]-1H-indo1-3-yl1-2,3-dihydroisoindo1-1-one, or a pharmaceutically acceptable salt thereof.

13. A method of treatment of pain, comprising administering an SOS1 inhibitor, or a pharmaceutically acceptable salt thereof, and/or Ras inhibitor, or a pharmaceutically acceptable salt thereof, as described in claim 1 in combination with an anti NGF antibody.

14. The method of claim 13 wherein the anti-NGF antibody is Tanezumab.

15. The method of claim 1 wherein pain is chosen from acute pain; chronic pain; inflammatory pain; nociceptive pain; neuropathic pain; hyperalgesia; allodynia; central pain; cancer pain; post-operative pain; visceral pain; musculo-skeletal pain; heart or vascular pain; head pain; orofacial pain; and back pain.

16. The method of claim 1 wherein pain is chosen from:
(a) acute pain and/or spontaneous pain,
(b) chronic pain and/or on-going pain,
(c) inflammatory pain,
(d) nociceptive pain,
(e) neuropathic pain,
(f) hyperalgesia,
(g) allodynia,
(h) central pain, central post-stroke pain, pain resulting from multiple sclerosis, pain resulting from spinal cord injury, or pain resulting from Parkinson's disease or epilepsy,
(i) cancer pain,
(j) post-operative pain,
(k) visceral pain,
(l) musculo-skeletal pain, myalgia, fibromyalgia, spondylitis, sero-negative (non-rheumatoid) arthropathies, non-articular rheumatism, dystrophinopathy, Glycogenolysis, polymyositis, or pyomyositis,
(m) heart or vascular pain, pain due to angina, myocardical infarction, mitral stenosis, pericarditis, Raynaud's phenomenon, scleredoma, or skeletal muscle ischemia,
(n) head pain,
(o) orofacial pain, and
(p) back pain, bursitis, menstrual pain, referred pain, trigeminal neuralgia, hypersensitisation, pain resulting from spinal trauma and/or degeneration or stroke.

17. The method of claim 16, wherein the inflammatory pain is arthritic pain, pain resulting from osteoarthritis or rheumatoid arthritis, pain resulting from inflammatory bowel diseases, psoriasis, or eczema.

18. The method of claim 16, wherein the neuropathic pain is painful diabetic neuropathy or pain associated with post-herpetic neuralgia.

19. The method of claim 16, wherein the visceral pain is digestive visceral pain non-digestive visceral pain, pain due to gastrointestinal disorders, pain resulting from functional bowel disorders, pain resulting from inflammatory bowel diseases, pain resulting from dysmenorrhea, pelvic pain, cystitis, interstitial cystitis, or pancreatitis.

20. The method of claim 16, wherein the head pain is migraine, migraine with aura, migraine without aura cluster headache, or tension-type headache.

21. The method of claim 16, wherein the orofacial pain is dental pain, temporomandibular myofascial pain, or tinnitus.
